(19) United States
(12) Patent Application Publication
(10) Pub. No.: US 2026/0144697 A1
(43) Pub. Date: May 28, 2026

(54) ROBOTIC WALKER FOR GAIT AND POSTURAL STABILITY ASSISTANCE WITH A HIGH LEVEL OF AUTONOMY AND SAFETY

(71) Applicant: Robility, Inc., Palo Altlo, CA (US)

(72) Inventors: Mojtaba Sharifi, Palo Alto, CA (US); Alexander Phillip Martin-Ginnold, Palo Alto, CA (US)

(21) Appl. No.: 19/268,545

(22) Filed: Jul. 14, 2025

Related U.S. Application Data
(60) Provisional application No. 63/724,948, filed on Nov. 26, 2024.

(51) Int. Cl. A61H 3/04; G16H 40/63

(52) U.S. Cl. CPC A61H 3/04; G16H 40/63; A61H 2003/043; A61H 2201/0161; A61H 2201/1207; A61H 2201/163; A61H 2201/1638; A61H 2201/1642; A61H 2201/5046; A61H 2201/5061; A61H 2201/5079; A61H 2201/5092; A61H 2230/62

(57) ABSTRACT

A device may include a rigid frame having a left side and a right side. A device may include a caster wheel attached to a front portion of each of the left and right sides of the rigid frame. A device may include a motorized wheel assembly attached to a rear portion of each of the left and right sides of the rigid frame, each motorized wheel assembly comprising an electric motor with a driven output coupled to a mecanum wheel configured to provide omnidirectional movement. A device may include sensors for detecting rotational position and speed of the electric motors. A device may include one or more cameras oriented toward a user of the walker configured to monitor user posture, gait, and balance.

### CROSS-REFERENCE TO RELATED APPLICATION

This application claims the benefit of U.S. Provisional Application No. 63/724,948, filed Nov. 26, 2024, which is incorporated herein by reference in its entirety.

### BACKGROUND

Falls are extremely common and often go unreported with as many as a third of individuals over 65 falling once a year. This group requires more attentive care and is expected to become a larger share of the population, creating a larger demand for mobility assistance. The Centers for Disease and Control Prevention states that around 12.1% of adults in the US suffer from some form of disability affecting mobility. This increased demand for mobility assistance will continue to put a strain on the healthcare professionals to assist the growing elderly population and other individuals with physical mobility impairments. Assistive devices are solutions to this need.

The type of assistive device is determined at the individual level by a trained medical professional and can range from wheelchairs, for those with extremely high assistance needs, to walkers and canes for those that require a lower level of assistance. Although walkers help people with mobility issues get some independence back, it has been known that these devices are not 100% effective and can lead to serious injuries caused by falling. Because of this, there has been considerable research focused on improvements to walkers in order to provide more personalized assistance for those who require it. These walkers have been called smart walkers because they significantly augment the walker's ability to safely assist the user in daily tasks, even for those with impaired cognitive and/or visual capacity. The main design goals of these smart walkers are to improve the safety of traditional walkers by enhancing the user's postural stability and navigating their surroundings.

The use of impedance/admittance control for a smart walker has proven to be extremely beneficial in being able to reliably predict the user's intent. An advantage of force-based impedance-based controllers is the capability to determine the user's intention using a force-torque sensor. This requires that the force-torque sensor be placed in a location where all the interaction forces of the user are transmitted through the sensor to the walker's body. The addition of sensors to prevent collision with objects in the environment can be performed by employing digital cameras, infrared, ultrasonic, or a combination of them. The main benefit of infrared and ultrasonic is that the data generated is easier to process since distance from the sensor is one of the captured parameters. A camera, on the other hand, is not able to provide distance readings as an output and needs to be further processed to determine objects in the walker's path. This data is then fed into the walker strategy to negotiate possible paths based on the user's intent.

The concept of potential field has been used for path planning in the fields of autonomous/semi-autonomous mobile robots and vehicles. This includes self-driving cars, un-manned aerial vehicles, autonomous ships, and tele-operated robots. In the case of a semi-autonomous system in which the desired trajectory is not predetermined by the system, the required inputs are the distance from the obstacle. These control systems work by monitoring the distance between the object and the robot itself but only activate when the conditions of a possible collision occur. A collision avoidance strategy that has been employed in naval vessels is in the use of potential fields that can alter a ship's desired direction by any obstacle entering different zones of control. These zones would be determined by the distance and ability of the vessel to navigate away from a possible collision. The avoidance algorithm won't be activated until the criteria for collision are met, which then would attempt to steer the vessel out of the collision path. The inclusion of a negotiating zone, in addition to an emergency zone, for obstacle avoidance can assist the autonomous system in generating smoother responses to possible obstacles. This method can also be extended to moving objects that enter the potential fields in which the object's speed can be determined.

The main goal of this work is to propose an intelligent control strategy and develop a mechatronic system to perform real-time motion planning for a motorized assistive walker based on the user and environment inputs. This trajectory shaping is conducted in response to the user's physical interaction and obstacle detection in space by integration of multiple sensors. A six-axis force-torque sensor is employed under the handlebar for the user's intention detection, a LiDAR sensor is used for scanning obstacles, and high-torque DC motors with motion feedback are utilized for all Mecanum wheels. An admittance controller is employed to generate the original motion (including the velocity di-rection and magnitude) in Cartesian space in terms of the user's physical interaction. By establishing different zones of control, the walker's avoidance algorithm can gently deliver the space feedback to the user by slowing (resisting) the movement in the direction of any obstacle within a specific range and increasing this resistance as the user gets closer to the obstacle. To this end, the potential fields are generated in real-time for scanned obstacles to regulate velocity modifications and update the motion trajectory using the proposed control scheme. This assistive walker will be helpful for the personalized locomotion of individuals with different levels of physical, visual, and mental impairments

People with physical disabilities and chronic conditions of-ten rely on assistive tools for their daily activities. Enhancing the affordability, design, and comfort of these devices can significantly improve their practicality for home use and enhance the life of individuals in need. Despite mechanical advancements in traditional walkers, they have not fully integrated robotic capabilities. This research aims to address this gap by providing practical solutions to a range of motor impairment challenges and developing a robotic walker with an optimum design for stability. The innovative walker features a 4-wheel structurally stable design, supported by the fundamental implementation of a Walker Tipping Index (WTI). Additionally, it includes motion planning based on the user's intention, collision detection, and fall prevention, all with the goal of enhancing the walking experience for individuals with motor impairments. The design of the walker chassis prioritized postural stability in all directions, utilizing a WTI calculator to determine overall dimensions while ensuring the index remained below 0.5 in all directions (front-back, left-right). For the structural integrity of the design, Finite Element Analysis (FEA) was conducted for symmetric and asymmetric loading, modal analysis, and buckling analysis. Results indicated a factor of safety above 2 for all worst-case load-ing conditions, with a buckling load factor of 5.94 and a natural frequency optimized to exceed the motor's operational frequency. Fabrication of the walker utilized AISI 4130 steel tubes to minimize weight while maximizing strength. LiDAR and force Sensor, electronics, and battery mounting components were 3D printed using carbon fiber-infused PETG, and Aluminum 6061-T was used for the motor mounts which were manufactured using CNC milling. Finally, a comprehensive physical stability test in all directions was conducted to validate the WTI calculations and confirm the postural stability of the walker structure.

### SUMMARY

Assistive walkers are essential for many individuals who require geriatric care or mobility aids for day-to-day activities. The functionality of these devices is not able to provide the care that is needed for those with different levels of physical disabilities or who are visually or mentally impaired. Developing an autonomous powered walker that can receive a user's intent and assist them in navigating obstacles would allow more people to have access to enhanced mobility. In this work, a control strategy is designed for obstacle avoidance using potential field generation and admittance regulation. The mechatronics hardware and software are developed to generate and implement a continuous real-time motion trajectory for the smart walker in response to the user's interaction torque and scanned locations of obstacles in the environment. The integration of the mecanum wheels, DC motors, microcontrollers, a rotating LiDAR scanner, and a mini-PC powered by an onboard battery allowed for the evaluation and testing of the obstacle avoidance algorithm and admittance control in real time. The experimental results showed that the autonomous walker can generate desired trajectories in response to the user's interaction using the admittance controller and navigate around the obstacles with real-time velocity updates for the motorized wheels.

Motor impairment in the elderly is attributable to an increase in muscle fatigue, loss of vision, and a loss of balance; all of which occur as the human body deteriorates with age. Assistive devices can help these individuals with daily living activities and improve their quality of life. Accordingly, walkers have entered the market with a variety of designs to provide elderly patients with some tools to help combat mobility-related hazards that accumulate as the body ages. In more recent efforts that this review will analyze, data-driven designs have been employed to solve more narrowly scoped mobility problems to move away from the idea of a general-purpose walker. This development should better suit the needs of individuals who may require walkers for different purposes. It was observed that robotic assistance has tried to solve this problem by introducing intelligence for fall prevention and object detection through sensing modules which in real time analyze users' GAIT performance and sense the objects around. In a research study, the mechanical instability in the design of the assistive walker can be solved by thoroughly understanding the design trends and stability calculation. A robotic motorized walker with a stable mechanical design for postural stability can solve the problem of motor impairment.

Considerable attempts have been made in the past to solve the problem of motor impairment by using stick-like supporting objects known as walkers. Over time, these designs became more reliable, but the fundamental concept remained mechanical with little to no use of electronics. In order to create a mechanically stable design with an intelligent sensing module that can make the walker robotic and more useful for a range of motor impairment issues, our research project aims to solve this problem. The desired result of research in this field is to uncover and experiment pragmatic solutions by adding to (or modifying) a stable, 4-wheel, electrical design to provide enough comfortable mechanical adjustments, and for sensors and controllers to aid in collision detection, fall recovery, and prevention, and to improve the general walking experience.

Originally, assistive canes, initially crafted from wood to address motor impairment, evolved into walker-cane hybrids, providing lateral stability lacking in traditional canes. Traditional walkers, with four foldable legs, offered structural triangulation for enhanced stability and feedback. Front-wheeled walkers aimed to improve mobility, but their design limitations persisted. The introduction of rollators, particularly upright walkers, provided sturdier support without compromising the user's back. To enhance mechanical and postural stability, motorized wheels, and a sensing module for fall prevention were incorporated into upright walkers. Finkel's research highlighted critical walker failure modes, with motor-controlled wheels addressing sliding and brake failure, while tipping required mechanical solutions. Walker design principles, rooted in gait analysis and the Walker Tipping Index (WTI), informed quantitative evaluations. Conventionally, WTI and gait analysis for stability have been integrated, and there has been focus on battery efficiency and weight reduction, emphasizing usability and cost-effectiveness.

The landscape of walker optimization extends beyond mobility to encompass design features that unlock a broader range of mobility options for users. Me'de'ric pioneers a versatile design, enabling users to sit freely in diverse environments. Other conventional systems broaden the operable terrain of walkers by introducing rotary magnetorheological brakes and direct current motors, forming an active-passive hybrid actuator system facilitating easier traversal across varying inclines. And other conventional systems pivot towards ergonomics, concentrating on reducing upper body strain in mobility scenarios where lower body mobility is compromised. The domain of robotic walker control witnesses noteworthy innovations. Other systems have contributed towards a novel robotic walker characterized by omnidirectional assistance, body weight distribution support, and hip rotation capabilities, promising an enhanced rehabilitative experience. Conventional technologies integrate omnidirectional wheels, a PID-BVE controller, and an obstacle detection system to empower the walker to detect and divert users from obstacles, exemplifying the potential of advanced control mechanisms.

Sensor-based approaches emerge as a pivotal paradigm, significantly influencing the evolution of walker designs. Other systems leverage laser, ultrasonic, vision-based, and Leap motion sensors to infer control mechanisms, predict gait trajectories, and detect knee positions. This sensor-driven augmentation enhances the overall utility and effectiveness of walker designs. A particularly note-worthy development is the sensor-based guidance system pre-sented in a study, where the system empowers visually impaired walker users by associating sensors with a GUI on a tablet. Users can directly interface with sensors, react to obstacles, and navigate along planned paths, showcasing a paradigm shift towards user empowerment in tandem with sensor-driven advancements. In this study, we developed the structure of an intelligent robotic walker through a systematic process. The initial step involved calculating the fundamental dimensions using a Walker Tipping Index (WTI) spreadsheet calculator. These dimensions were optimized to maintain the WTI below 0.5 for both forward-backward and leftward-rightward tipping, ensuring a stable de-sign. To select the force sensor, the maximum body weight of a 100 kg person applying full load onto the walker was considered. After extensively comparing various force-torque sensors, “ATI mini-58” was selected due to the loading limits required for this application (postural support). The structural design was manufactured using AISI 4130 pipes, selected after rigorous weight optimization, and postural and structural stability considerations through various types of symmetric-asymmetric, buckling, and modal analysis. These analyses aimed to enhance the efficiency and robustness of the walker's structure. The manufacturing process involved Tungsten Inert Gas (TIG) welding for the structural components, while 3D printing was used for the electronic enclosure and LiDAR mounts, and Computer Numerical Control (CNC) machining for motor mounts. A physical stability test was conducted to validate the WTI analysis and confirm the postural stability and structural strength of the walker.

In some aspects, the techniques described herein relate to a walker including: one or more LiDAR sensors configured to perform range finding and obstacle detection; and an autonomous walker controller configured to: generate a motion trajectory for electric motors based on a user's surroundings, body posture, and intended movement; automatically guide the walker around obstacles with the motion trajectory, even if in conflict with the intended movement; guide the walker into a more supportive position with the motion trajectory if the user is at imminent risk of a fall; stop all movement if a use scenario becomes unsafe; identify a fall by estimating a relative position of the user's center of mass; and generate a profile of the user's habitual movements and preferences to personalize the generated motion trajectory.

In some aspects, the techniques described herein relate to a walker, further including a rigid frame having a left side and a right side.

In some aspects, the techniques described herein relate to a walker, further including a caster wheel attached to a front portion of each of the left and right sides of the rigid frame.

In some aspects, the techniques described herein relate to a walker, further including a motorized wheel assembly attached to a rear portion of each of the left and right sides of the rigid frame, each motorized wheel assembly including an electric motor with a driven output coupled to a wheel.

In some aspects, the techniques described herein relate to a walker, further including sensors configured to detect rotational position and speed of the electric motors.

In some aspects, the techniques described herein relate to a walker, further including an adjustable armrest with an integrated grip and a plurality of force sensors attached to each of the left and right sides of the rigid frame.

In some aspects, the techniques described herein relate to a walker, further including brake levers physically coupled to brakes on wheels and sensors configured to detect an amount of brake actuation.

In some aspects, the techniques described herein relate to a walker, further including one or more cameras oriented toward the user to monitor user posture, gait, and balance.

In some aspects, the techniques described herein relate to a walker, further including one or more cameras oriented toward an environment surrounding the walker to monitor type and position of obstacles.

In some aspects, the techniques described herein relate to a walker, further including user interfaces for control including one or more of: screens to display control and user interfaces, physical buttons, microphones, speakers, and LED lights.

In some aspects, the techniques described herein relate to a walker, further including a seat permanently fixed between left and right sides of a rigid frame.

In some aspects, the techniques described herein relate to a walker, further including a stowable seat permanently fixed between left and right sides of a rigid frame.

In some aspects, the techniques described herein relate to a walker, further including stowable foot rests configured to support weight of the user while standing on the walker.

In some aspects, the techniques described herein relate to a walker, further including the stowable foot rests.

In some aspects, the techniques described herein relate to a walker, wherein the autonomous walker controller is further configured to monitor sensor data to map surroundings of the walker and track body posture of the user.

An upright wheeled walker with automated powered assistance, the walker comprising: a rigid frame having a left and right side; a caster wheel attached to the front of each of the left and right sides; a motorized wheel assembly attached to the rear of each of the left and right sides, the assembly consisting of an electric motor with the driven output coupled to a wheel; sensors for rotational position and/or speed of the electric motors; an adjustable armrest with integrated grip and a plurality of force sensors attached to each of the left and right sides; brake levers physically coupled to brakes on the wheels; sensors to detect the amount of brake actuation; one or more cameras oriented toward the user; one or more camera oriented toward the environment; and one or more LiDAR sensor.

User interfaces for control consisting of one or more of the following, one or more screens to display control and user interfaces; one or more physical buttons; one or more microphones; one or more speakers; and one or more LED lights.

An autonomous walker controller to: monitor sensor data to map the surroundings of the walker; monitor sensor data to track the body posture of the user; and monitor sensor and user interface data to track the intended movement of the user.

Further, generate a motion trajectory for the electric motors based on the user's surroundings, body posture, and intended movement. The motion trajectory may automatically guide the walker around obstacles, even if in conflict with the intended movement. The motion trajectory may guide the walker into a more supportive position if the user is at imminent risk of a fall. The walker may stop all movement if the use scenario becomes unsafe. The walker may identify a fall by estimating the relative position of the user's center of mass. Generate a profile of the users habitual movements and preferences to personalize the generated motion trajectory

The walker from claim one, with the addition of a seat permanently fixed between the left and right sides of the rigid frame

The walker from claim one, with the addition of a stowable seat permanently fixed between the left and right sides of the rigid frame

The walker from claim one, with the addition of stowable foot rests to support the weight of the user while standing on the device.

### BRIEF DESCRIPTION OF FIGURES

The disclosed aspects will hereinafter be described in conjunction with the appended drawings, provided to illustrate and not to limit the disclosed aspects, wherein like designations denote like elements.

FIG. 1 illustrates the walker in a typical use environment with visualizations of the LiDAR and camera sensing capabilities

FIG. 2A illustrates the mechanical components and features of the walker.

FIG. 2B represents another embodiment of the walker.

FIG. 3A represents the coordinate system and key dimensions used when describing the walker of embodiment shown in FIG. 2A.

FIG. 3B represents the coordinate system and key dimensions used when describing the walker of embodiment shown in FIG. 2B.

FIG. 4 represents an illustration of the BoS for a traditional walker and the presented walker.

FIG. 5A illustrates the walker's mechatronic components and their locations.

FIG. 5B illustrates another embodiment of the walker, wherein the height of the walker's upper portion is controlled and adjusted by electric motors.

FIG. 6 represents a schematic diagram of the mechatronic and control design of the walker.

FIG. 7 shows a visualization of the expected output of the FCM.

FIG. 8 is a schematic of a smart walker with sensors, actuators and controller box.

FIG. 9 defines potential field zones for the autonomous assistive walker to perform stationary obstacle avoidance.

FIG. 10 illustrates the fully integrated walker with mechatronic components.

FIG. 11 represents the components and configuration schematic of the walker's mechatronic system used to receive sensory information and control the motors.

FIG. 12 illustrates an experimental result of the processed data from the RP LiDAR showing scanned obstacles around the sensor.

FIG. 13 shows the architecture of the developed mechatronic systems, the executed tasks, and signal transmission between different components.

FIG. 14 illustrates the force and torque values measured by the ATI sensor under the handlebar.

FIG. 15 represents the obtained values for the desired velocity were processed and obtained.

FIGS. 16, 17, 18 and 19 demonstrate the results of speed variation in Cartesian and wheels spaces.

FIG. 20 represents the calculated tipping forces in reference to a Free Body Diagram.

FIG. 21 shows the Final walker design with force sensor only supported by user's applied force via the handlebar, with added cross pipes for a stiffer structure that has a high natural frequency.

FIG. 22 is a rendered CAD model of the walker with omnidirectional wheels, hub motors, electronics enclosure containing battery and electronics.

FIG. 23(a)-(b) shows comparison of natural frequency between old and new design with added pipes for structural rigidity.

FIG. 24(a)-(b) shows a comprehensive structural analysis where there is certain force acting downwards on the handlebar.

FIG. 25(a)-(b) represents additional analysis to assess the rigidity of the structure where (a) Buckling Analysis performed on handlebar when it bears triple the person's weight.

FIG. 26 represents Postural stability test on the walker structure to validate the WTI calculations and structural strength.

FIG. 27 shows the final assembly and manufactured walker with all the components.

### DETAILED DESCRIPTION

The present disclosure can be understood more readily by reference to the instant detailed description, examples, and claims. The present disclosure is not limited to the example embodiments and/or methods disclosed herein, which may, of course, vary. It is also to be understood that the terminology used herein is for the purpose of describing particular embodiments only and is not intended to be limiting. Unless defined otherwise, all technical and scientific terms used herein have the same meaning as commonly understood by one of ordinary skill in the art.

The present application relates to a robotic walker for gait and postural stability assistance with a high level of autonomy and safety. The design improves stability to minimize the risk of falling compared with traditional walking frames and rollators. The walker's mechanical design is optimized for stability based on the walker Tipping Index (WTI), a metric of the likelihood that a walker will fall in each direction. The walker uses an array of sensors and actuators. Detect the user's movements and interaction forces to identify their behavior and intention. Monitor the user's surroundings for obstacles and hazards. Automatically maneuver the walker to support the user during a fall or to avoid obstacles. The sensors include tension-compression load cells in the handles to measure the user's physical interaction with the walker, a LiDAR sensor to measure the walker's distance from its surroundings, and cameras to provide additional detail of the walker's surroundings and enable object recognition using image processing.

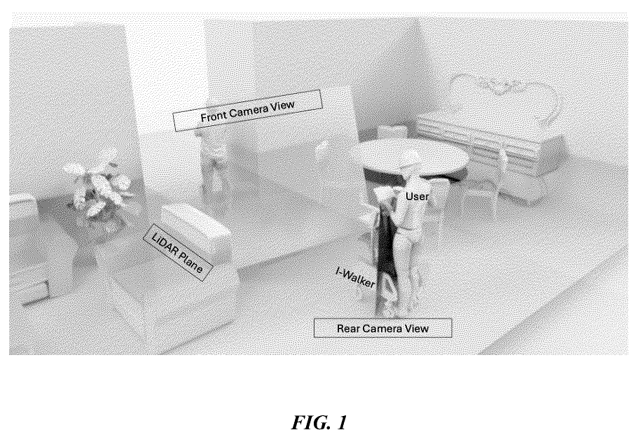

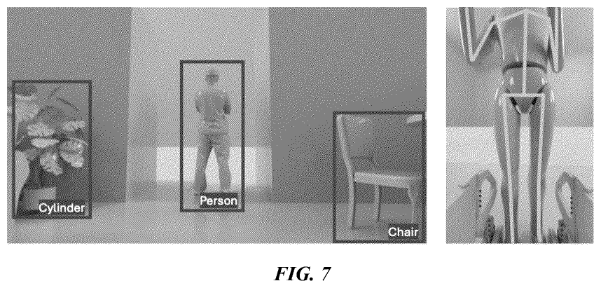

FIG. 1 illustrates the walker in a typical use environment with visualizations of the LiDAR and camera sensing capabilities. In particular, as shown in FIG. 1, the walker with a user and another person in a typical use environment. The optical sensing capability of the walker is illustrated. The LiDAR measurement plane intersects with the furniture and other people in the room. The distance to any object on this plane with a line of sight to the walker can be accurately measured to support environment mapping. The field of view of the front-facing camera is shown by the light shaded frustum. This camera is used for object detection and avoidance. The field of view of the rear-facing camera is shown in the dark shaded frustum. This camera is used for pose and postural stability estimation. (See FIG. 7 for details on the camera views).

The walker's motorized wheels empower the user to walk with minimal energy by pushing both the walker and the user in the intended direction. This motorized walker can automatically adjust its speed and position to support the user during a fall or loss of balance and autonomously avoid obstacles detected by the sensors. The walker is powered by an on-board rechargeable battery. The walker includes a touchscreen, speaker, and microphone so the user can interact with and control the walker.

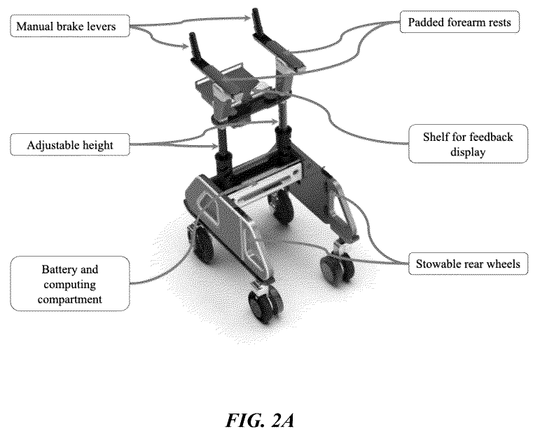

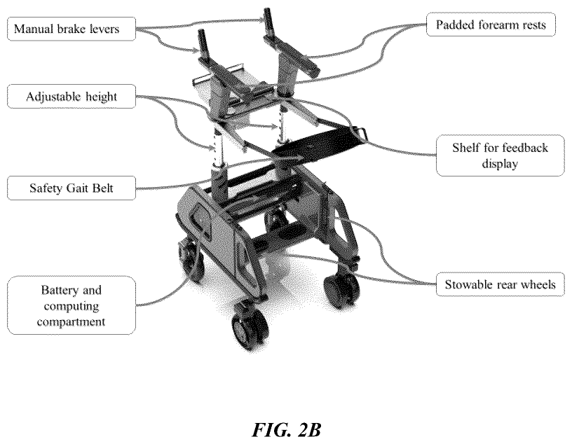

Next, FIG. 2A highlights the key mechanical and design features of the walker shown in FIG. 1. FIG. 2A illustrates the mechanical components and features of the walker. The walker is adjustable to suit a range of users. The manual brakes allow the walker to be safely operated, even if the battery runs out.

Next, FIG. 2B represents another embodiment of the walker. As shown in FIG. 2B, a safety gait belt is connected to the upper section to further maintain the user's postural stability in addition to motion planning for fall prevention. This feature will minimize the risk of falls even in cases where the walker's motion planning cannot resolve the postural instability or the user loses the grasp and support from the handles and forearm rests.

This gait belt is also used to support the user's body and assist in sit-to-stand and stand-to-sit motions, to be moved together with the forearm rests. This will be provided by moving the upper portion of the walker using electric motors up and down as much as needed by a button.

### Mechanical Design

The mechanical structure of the walker, as shown in FIG. 2A, was designed to optimize the WTI, a calculated metric based on the geometry of a walker and the forces the walker is subjected to that can predict the walker's likelihood of tipping.

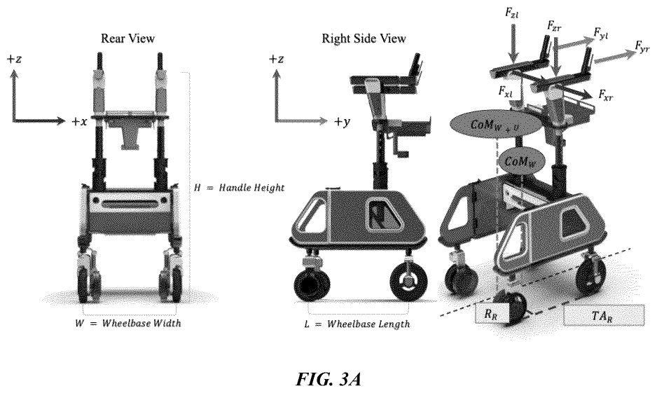

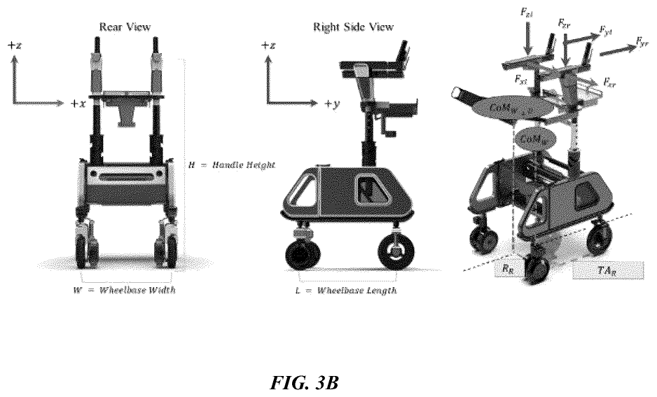

Next, FIG. 3A represents the coordinate system, interaction forces, and dimensions of the walker as illustrated in FIG. 2A. FIG. 3A represents the coordinate system and key dimensions used when describing the walker. The CoM_{W} refers to the center of mass of the walker alone, while CoM_{W+U} describes the shared center of mass of the walker and the user. The tipping axis TA_{R} and R_{R} are shown for the rightward direction. Whereas, FIG. 3B represents the coordinate system, interaction forces, and dimensions of the walker as illustrated in FIG. 2B.

The WTI can be calculated for tipping in the anterior-posterior (AP) or medial-lateral (ML) directions, where tipping is assumed to occur about an axis connecting two adjacent wheels, TAₛ, where the subscript s is replaced with the direction of tipping associated with the axis. A subscript L indicates leftward tipping, R indicates rightward tipping, A indicates forward tipping, and P indicates backward tipping. The general equation for WTI is shown in Equation 1, where Fₜ includes any horizontal force acting perpendicular to the tipping axis, Fᵥ is any vertical force applied to the walker including its own weight, and Rₛ is the perpendicular distance of the center of pressure to TAₛ.

$${WTI}_{s} = \frac{\sum {F}_{t} \cdot H}{\sum {F}_{v} \cdot {R}_{s}} \qquad ( 1 )$$

The walker has a height, wheelbase, and handle positions designed to minimize the WTI. The walker is optimized to have a WTI of less than 0.5 in every direction. This means the walker is less likely to tip in any direction than a standard aluminum walker, enhancing users' safety and feeling of security.

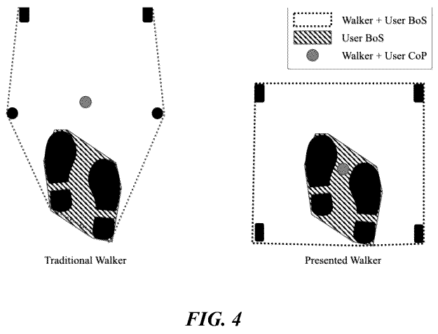

The walker encourages the user to walk upright within its wheelbase. The upright design of the walker allows the user to rest their forearms on the device during use to support their weight while enabling them to keep their center of mass within their unassisted base of support (BoS_{U}), as shown in FIG. 4.

FIG. 4 represents an illustration of the BoS for a traditional walker and the presented walker. If the CoP moves outside of the BoS a fall is likely. If the CoP falls outside of the user's BoS during walker use they can be supported by the walker. However, this position leaves them vulnerable to a fall if the walker cannot support them, for example if they lose their grip on the handles. The walker's design keeps the user in an inherently stable position

In contrast, traditional walkers require users to lean forward to support their weight, which places their individual center of mass outside of BoS_{U} and makes a fall likely if the user loses their grip on the walker. The upright walking position of the walker is more ergonomic and reduces the likelihood of strain or injuries to the wrist or hand.

### Mechatronic System Design

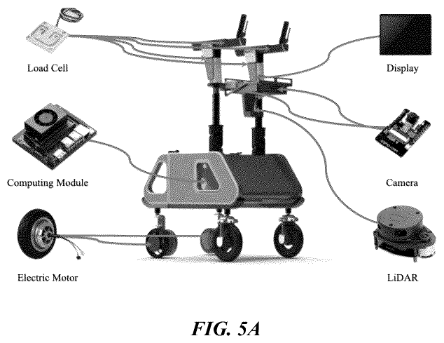

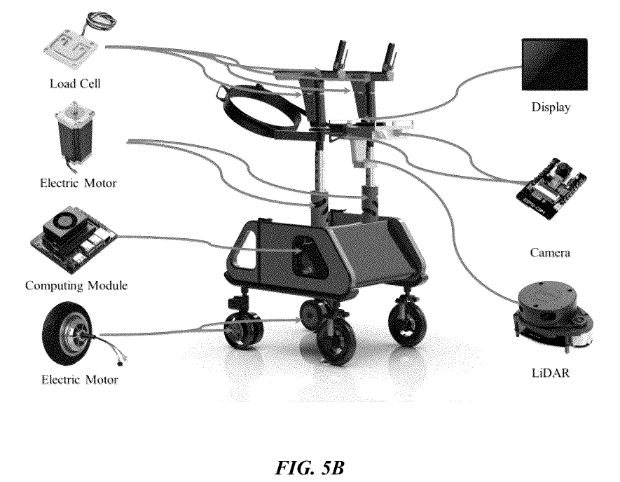

FIG. 5A shows the location of the motors and other electronic components on the walker as illustrated in FIG. 2A. Unlike passive walkers, the walker disclosed herein incorporates lightweight motors with enough power to carry the entire weight of the user

FIG. 5A illustrates the walker's mechatronic components and their locations. The final mounting points of the cameras, LiDAR, and load cells are subject to optimization efforts during this Phase I grant. The computing module is held in a secure compartment low in the walker to keep the COM low. Whereas, FIG. 5B illustrates another embodiment of the walker, wherein the height of the walker's upper portion is controlled and adjusted by electric motors. Using this feature, the gait belt and the forearm rests are movable up and down as much as needed by a button to support the user's body and assist in sit-to-stand and stand-to-sit transitions

The motors can propel the walker according to the user's interaction forces and adjust the user's walking path to the safest and most stable option. The walker incorporates foot supports that can be deployed so the device can be used as an assistive scooter if the user becomes fatigued while walking. This allows users to increase their activity range and remain active without worrying about becoming too tired during activity and not being able to return home. The position of the motors in the rear wheels allows the walker to pivot around the user during sharp turns.

The walker uses four single-axis load cells to measure user interaction forces. The mechanical interface and positioning of these load cells in each handle makes it possible to directly measure the y and z components of the user's interaction force and estimate the interaction torques about the y and z axes. The sensors are positioned to capture all interaction forces along their axis of measurement for each handle (F_{yl}, F_{yr}, F_{zl}, F_{zr}, FIGS. 3A-B). To maintain measurement accuracy, the mechanical design supports the load cell such that out-of-axis forces do not load the sensor, but in-axis forces are supported entirely by the sensor.

The walker incorporates three optical sensors to monitor the user and their environment: one LiDAR and two visible-light cameras. All three optical sensors are visualized in FIG. 1. The LiDAR sensor measures the distance to the nearest object in a horizontal plane around its center (its “detection plane”). This sensor is central to the walker's obstacle avoidance and path-planning ability. The LiDAR must be mounted securely to the walker to minimize its movement relative to the walker so distance measurements are consistent. The LiDAR must be placed to minimize the amount of its detection plane obstructed by the walker. The LiDAR must be placed at a height that maximizes the number of common obstacles, such as furniture and people, that intersect with its detection plane. The first camera faces forward and is used to detect and identify objects ahead of the walker. This camera supplements the LiDAR sensor by providing insight into the 3D scene ahead of the walker, so it is placed and aimed to ensure reliable detection of obstacles that cannot be detected by the LiDAR, such as stairs and curbs below the LiDAR detection plane, and large tabletops or objects hanging from the ceiling above the LiDAR detection plane. The second camera faces rearward, toward the user, and is used to estimate the user's body posture. is placed to maximize its view of the user. Each optical sensor is placed in an area that is unlikely to be obscured by the user's clothing or belongings during typical use.

### Motion Planning and Control

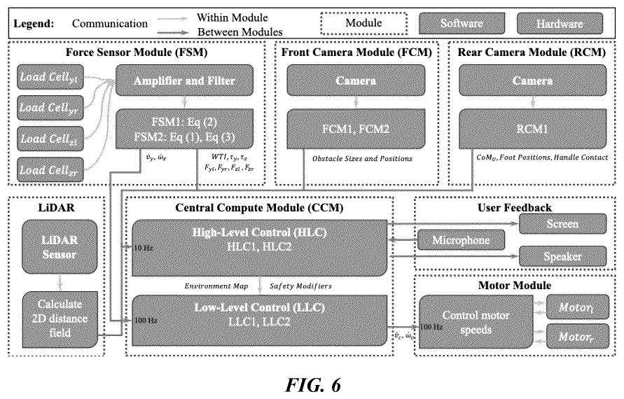

FIG. 6 shows a schematic representation of the walker modules as incorporated in the walker illustrated in FIG. 2A. FIG. 6 represents a schematic diagram of the mechatronic and control design of the walker. Sensor fusion and robotic control strategies are implemented across a Force Sensor Module (FSM), a Front Camera Module (FCM), a Rear Camera Module (RCM), and a Central Compute Module (CCM).

The FSM, FCM, RCM, CCM, and Motor Module are the focus of this Phase I grant. The LiDAR module is an off-the-shelf part. The frequencies listed at the termination of some arrows are the minimum communication frequencies that are critical to the responsive and safe operation of the walker.

For overall system control, the CCM balances inputs that monitor user intention, user stability, and obstacle locations to generate and output the ideal commands to the motors. The FSM, FCM, and RCM pre-process the raw sensor data as much as possible before transmitting results to the CCM. This arrangement allows the cost and computing power of the CCM to be reduced by reducing the number of computations it must perform for each control update.

Force Sensor Module: The FSM translates raw load cell electrical signals into the user's interaction forces, the user's intended movement for the walker, and multiple measures of postural stability. The conversion from digital signal to force for each load cell is calibrated with known weights. The module implements two further algorithms using the force measurements as inputs to generate control signals for the walker.

FSM1: The force sensor module converts the user's interaction forces to the user's intended motion for the walker. This algorithm takes the form of the differential equation shown in Equation 2, where \dot{v}_{y} is the intended linear acceleration, \dot{ω}_{z} is the intended angular acceleration, v_{y} is the linear velocity, and ω_{z} is the angular velocity. The functions G and H include constants to control the sensitivity of the walker's controls to the different interaction forces. These constants are customizable so each user can personalize the behavior of their device. Solving Equation 2 produces the users intended velocities v_{y} and \dot{ω}_{z} A key result of TO2 for this module is to determine the specific functions G and H from Equation 2.

$$( \begin{matrix}{\dot{v}}_{y} \\ {\dot{ω}}_{z}\end{matrix} ) = G ( {F}_{y l} , {F}_{yr} , {F}_{zl} , {F}_{zr} ) ( \begin{matrix}{v}_{y} \\ {ω}_{z}\end{matrix} ) + H ( {F}_{yl} , {F}_{yr} , {F}_{zl} , {F}_{zr} ) \qquad ( 2 )$$

FSM2: The second computation step implemented by the force sensor module estimate the WTI in the AP directions, WTI_{A} and WTI_{P}, and estimate the user's interaction torques about the y- and z-axes, τ_{y} and τ_{z}. The y and z components of force are measured directly by the FSM, so WTI_{A} and WTI_{P} can be calculated by Equation 1. Each force sensor pair is used to approximate the torque about a perpendicular axis, τ_{y} from the z-axis forces and τ_{z} from the y-axis forces, as shown in Equation 3. The calculated WTI and torque estimates are transmitted to the CCM for use in robot control. A key result of TO2 for this module is to determine ideal values for the specific functions 1 and m from Equation 3.

$$( \begin{matrix}{τ}_{y} \\ {τ}_{z}\end{matrix} ) = ( \begin{matrix}m ( {F}_{zl} , {F}_{zr} ) \\ l ( {F}_{yl} , {F}_{yr} )\end{matrix} ) \qquad ( 3 )$$

Front Camera Module: The FCM interprets results from the front-facing camera to perform obstacle detection. FIG. 7 shows a visualization of the expected output of the FCM. FIG. 7 represents a simulated camera view from the FCM and RCM with the post-processing overlaid. In the left image, the FCM view highlights regions to show the detection of specific objects including a chair and a person, and the cylinder category of unidentified object. In the right image, the RCM identifies the position of each of the body segments (shown in green solid lines) and several reference landmarks on the walker frame (shown in red dotted lines).

FCM1: The module will perform lightweight image segmentation and object detection using an AI model such as Ultralytics YOLO. The FCM will perform image segmentation to identify the borders of areas containing a single object or obstacle and areas containing no objects. Object recognition will determine whether the detected border contains a person or a non-person object. Within the non-person objects, a small set of common obstacles (e.g., stairs, chairs, tables) are identified, while the rest are categorized by their shape (e.g., box, ball, cone, pole).

FCM2: The distance to and size of each person and non-person are estimated based on the camera lens position and field of view. Fixed visual landmarks on the walker's frame are included in the camera field of view to provide a reference for calculating the distance and size of other objects in the frame. The typical size of each common object detectable by the model is stored and used as an additional reference value in the distance and size estimation. The position and size estimations of all detected people will always be transmitted to the CCM. The position and size of all other objects are sorted by increasing distance from the walker, and in cases when many objects are detected, only data for the nearest objects are transmitted.

Rear Camera Module: The RCM interprets results from the rear-facing camera to track the user's posture and interaction with the walker. FIG. 7 shows a visualization of the expected output of the RCM.

RCM1: The module will implement a pose estimation AI model such as Ultralytics YOLO. Based on the detected pose and fixed visual landmarks on the walker's frame included in the camera field of view, the microcontroller will estimate the pose in 3D space. This 3D pose is used to estimate the user's COM, the position of each foot relative to the walker, and whether the user is holding the walker handles. All three estimates are transmitted to the CCM for use in robot control. Key results of TO2 for this module are to train an appropriate pose estimation AI model; deploy it to a low-cost microcontroller; and develop the CoM, foot position, and handle contact estimation algorithms.

Central Compute Module: The CCM receives control inputs from the FSM, FCM, RCM, and LiDAR and performs sensor fusion to generate controls for the walker's motors. The compute module will implement a version of the Robot Operating System (ROS) to interface with each sensor module and model the robot's behavior. The computations in the CCM are broken into a high-level control (HLC) thread and a low-level control (LLC) thread, each with a different update rate, to allow the walker to remain responsive to user inputs while performing the more complex sensor fusion and AI processing steps.

High-Level Control: HLC will operate at a minimum of 10 Hz to compute estimations of the user's postural stability and map the environment around the walker.

HLC1: The first task of the HLC thread is to map the environment around the walker. The thread will perform simultaneous localization and mapping (SLAM) based on the LiDAR distance information. SLAM will produce a map of everything detected in the plane of the LiDAR sensor based on the point field output of the LiDAR module. The map is updated with each scan of the LiDAR. Once the map is created, the object information transmitted to the CCM from the FCM is placed within the map based on their estimated positions. If any camera-detected objects overlap with LiDAR-detected items on the map, the position of the camera-detected objects is updated to align with the LiDAR-detected items. The items detected by the camera are labeled in the SLAM map, and the relative importance of avoiding the object is associated with the position. For example, a person detected ahead of the walker would be rated as more important to avoid than a small box. All areas of the map that are unidentified are rated as equally important to avoid. When the map update is complete, it is used by the LLC thread to perform path planning and obstacle avoidance.

HLC2: The second task of the sensor fusion thread is to perform postural stability estimation based on the CoM_{U}, foot positions, and handle contact data from the RCM and the WTI and torque data from the FSM. The postural stability estimation is used generate “safety modifier” constants that are applied to the user's intended motion v_{y} and ω_{z} output from the FSM to generate a final, corrected motion output, v_{c} and ω_{c}.

There are many ways that the postural stability measurements will trigger corrections to the intended motion. If CoM_{U} moves too far behind the walker, it indicates that the user may be falling forward and pushing their arms out to catch themselves, so the walker should move forward more slowly. If the user's feet are positioned one ahead of the other, they are likely walking, and it is safe for the walker to move ahead. However, if their feet are planted next to each other, the walker should not move forward too quickly to help keep the user's CoM_{U} close to BoS_{U}. The same safeguards based on CoM_{U} and foot position are applied for backward movement.

These safeguards allow the walker to transition to acting as a stable, stationary support structure if the user is falling. However, if the user continues to apply more force to the handles while the walker is still, the walker may tip. The AP WTI is tracked during falls, and the walker's movement is adjusted to maintain the WTI below a level with a high risk of tipping. To adjust the WTI the walker will allow some movement in the direction of Fₜ (from Equation 1), which will in turn increase Rₛ and reduce WTI.

As an additional safeguard, the handle contact estimation data is tracked, and the walker's movement is stopped if the user loses contact with the walker for a given period. The handle contact is estimated by the RCM and based on the z-axis forces on the handles. The force and torque data from the FSM will be incorporated to directly detect fall scenarios. For example, if the force is backwards and with high torque about the z-axis, the user is likely falling backwards and to the side. In this situation, the walker will move with a turning maneuver to allow the user to better support themselves using the handles.

Low Level Control: LLC will operate at a minimum of 100 Hz to calculate the control signals for the motors. The task of the control thread is to convert v_{y} and ω_{z} output by the force sensor module to commands for the two motors that achieve those targets while adding safety and stability features.

LLC1: The walker will implement a potential field algorithm to gradually steer away from oncoming obstacles. The potential field algorithm will calculate the direction the user intends to move based on their input force and calculate if the intended trajectory intersects with an object. If the trajectory intersects with an object, the algorithm will steer the walker in the direction of the nearest edge of the obstacle. The walker will evaluate the space available between objects on the map and treat spaces smaller than the walker as obstacles. The amount of adjustment that the walker provides is proportional to the range of the obstacles, with a shorter range triggering a stronger correction. If the walker detects an obstacle within a very short range, it will prevent any further movement toward the obstacle. For any objects that are marked on the map as important to avoid by HLC the range at which potential field corrections will take effect is increased.

LLC2: The safety modifiers are applied to the user's v_{y} and ω_{z} inputs to scale them as needed to perform obstacle avoidance or fall prevention maneuvers.

### Data Collection

Data collected by each of the sensors, including interaction forces between the user and walker, the movement of the walker, the user's body position, and the mapped environment around the walker, is stored locally on the walker to allow locally deployed AI to provide personalized assistance profiles. The walker adjusts its assistance based on the user's gait patterns, postural stability, and frequently navigated routes. The walker is capable of transmitting anonymized data to a central repository to be used to study user gait and walker user patterns and develop improvements for the walker.

The mechatronic system development and components to implement the proposed control strategy are explained in this section. A rotating LiDAR sensor is utilized to observe the distance of objects around the walker to be used for the obstacle avoidance algorithm. A six-axis force-torque sensor is employed to measure the user's physical interaction in order to determine the user's intent (the desired direction and speed of motion).

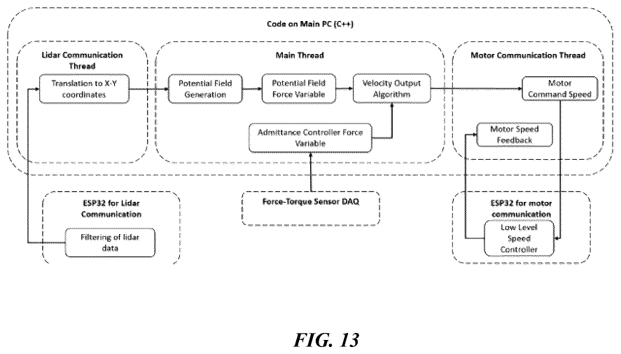

An admittance model is defined to generate the desired velocity vector in Cartesian space in terms of the measured interaction forces. This desired translation and rotational velocities are translated to the Mecanum wheels' space to be implemented by DC motors. FIG. 13 (discussed below) shows the architecture of the developed mechatronic systems, the executed tasks, and signal transmission between different components. An ESP32 microcontroller is dedicated to the LiDAR communication process and the data (angle and distance) received from the RP LiDAR sensor and sent to the thread to be used in the main C++ code for motion planning. The communication thread converts the data into a matrix of x-y obstacle locations and stores it until requested by the main thread (C++ code). This main thread generates the potential field, determines user intent (velocity vector in Cartesian space, Vₓ and V_{y}) based on force-torque measurement, and computes the desired speed of the walker by converting the Cartesian speed into motors' rotation using a matrix transformation according to the kinematics of Macanum wheels. The corresponding equations for these components of the proposed motion planning strategy are mentioned in the next sections. An ESP32 microcontroller receives the speed vector of wheel rotations and converts it into individual CAN commands to the DC motors.

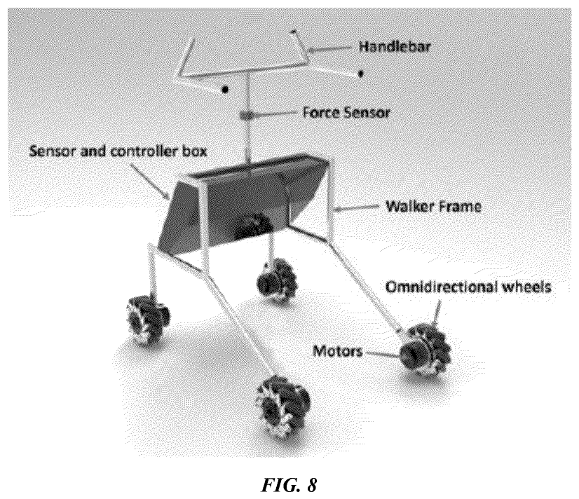

The walker structure is shown in FIG. 8 according to another example embodiment. FIG. 8 is a schematic of a smart walker with sensors, actuators and controller box. The walker represented in FIG. 8 includes four omni-directional wheels and DC motors, a force-torque sensor, LiDAR scanners, and a controller box that houses a mini PC, microcontrollers, a DAQ device, and a battery.

### Mecanum Wheel Kinematics

Having a 4-wheeled vehicle with mecanum wheels, one can derive the forward and backward kinematics of this assistive walker.

$$[ \begin{matrix}{w}_{1} \\ {w}_{2} \\ {w}_{3} \\ {w}_{4}\end{matrix} ] = \frac{1}{r} [ \begin{matrix}1 & - 1 & - ( a + b ) \\ 1 & 1 & ( a + b ) \\ 1 & 1 & - ( a + b ) \\ 1 & - 1 & ( a + b )\end{matrix} ] [ \begin{matrix}{v}_{x} \\ {v}_{y} \\ {w}_{z}\end{matrix} ]$$

In the above equation, a and b parameters are the distances of the wheels to the walker center in Cartesian x-y coordinates, wᵢ is the wheel speed while r is the radius of the wheels, w_{z} is the rotational speed of the walker around its z-axis, while vₓ and v_{y} are the translational velocity components of the walker along the x and y axes, respectively. This kinematic equation is used for the generation of velocity commands to the DC motors in terms of the desired Cartesian speed of the walker.

### Obstacle Avoidance Development

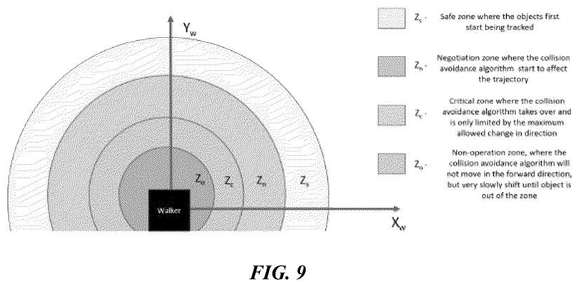

For obstacle avoidance, the generation of the potential field zones is conducted for online modification of the movement trajectory when there is any object in a collision path with the walker. The variation of these zones also prevents the movement trajectory from changing drastically in different directions. These potential field zones created for the walker can be seen in FIG. 9.

FIG. 9 defines potential field zones for the autonomous assistive walker to perform stationary obstacle avoidance. The size of the zone radius RZ where specified based on the walker's width w and Dmax as the maximum distance traveled over the sampling rate dt.

The trajectory planning is limited by the maximum distance traveled between samples. The obstacle avoidance inside the negotiation zone is adjusted based on the walker's width and the potential field values defined the below equation. The variable potential field value (PV F) is the magnitude of the potential field around the walker at a given distance.

$$[ \begin{matrix}{v}_{{x}_{p} f} \\ {v}_{{y}_{p} f}\end{matrix} ] = [ \begin{matrix}{v}_{{y}_{m} ax} \cdot \frac{{PFV}_{{RZ}_{measured}} - {PFV}_{{RZ}_{n}}}{{PFV}_{{RZ}_{n}} - {PFV}_{{RZ}_{c}}} \cdot DV \\ {v}_{{x}_{m} ax} \cdot \frac{{PFV}_{{RZ}_{measured}} - {PFV}_{{RZ}_{n}}}{{PFV}_{{RZ}_{n}} - {PFV}_{{RZ}_{c}}} \cdot DV\end{matrix} ]$$

The direction value (DV) is positive or negative depending on the slope of the potential field, and vₓpf and v_{y}pf are the velocity outputs generated by the potential field.

The potential field generation is based on a heat map of the obstacles inside of RZₛ. This creates a parabolic repulsive field with a value of 1000 at the obstacle and a value of 0 at a distance of RZ₀ from the obstacle according to the below equation:

$$PotentialMatrix = 1000 - ( ( {x}^{2} + {y}^{2} ) / {RZ}_{n}^{2} ) \cdot 1000$$

where y and x range from 0 to RZ

### Admittance Controller

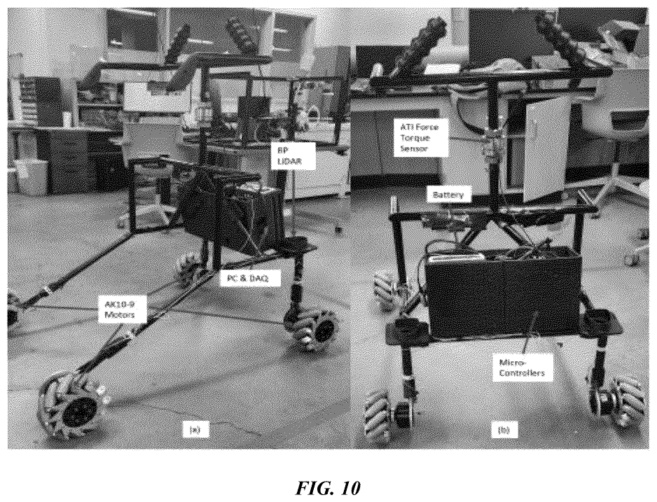

An admittance controller is developed and tested for the walker to generate the user's desired speed based on the interaction force and torque measured by the ATI sensor as shown in FIG. 10. The admittance control includes four variables including desired velocities in x and y directions, the rotational desired velocity, and the user balance variable. These translational and rotational velocities are determined by the below equation in terms of the interaction torque or forces (Tᵢ for i=z, x, or y). The inputs of this admittance model are the interaction torque in z direction for the axial rotation and the interaction forces in x and y directions for planar translation.

$$[ \begin{matrix}{T}_{low} < {T}_{i} < {T}_{high} : {V}_{i} = \frac{( {T}_{i} ) abs ( {T}_{high} - {T}_{i} )}{( {T}_{high} )} \cdot {V}_{{i}_{max}} \\ {T}_{low} > {T}_{i} ‖ {T}_{i} > {T}_{high} : {V}_{i} = 1 \\ abs ( {T}_{i} ) > {T}_{max} : {V}_{i} = 0\end{matrix} ]$$

The values of T_{control} and Tₘₐₓ are determined experimentally for each user of the walker. The user balance variable Sᵤₛₑᵣ is defined to determine user stability according to the below equation:

$$[ \begin{matrix}{S}_{min} < {Fz}_{user} < {S}_{{control}_{l}} : {S}_{user} = 1 + \frac{{S}_{{control}_{h}} - {Fz}_{user}}{{S}_{{control}_{l}} - {S}_{min}} \\ {S}_{{control}_{l}} < {Fz}_{user} < {S}_{{control}_{h}} : {S}_{user} = 1 \\ {S}_{{control}_{h}} < {Fz}_{user} < {S}_{max} : {S}_{user} = 1 + \frac{{S}_{max} - {Fz}_{user}}{{S}_{max} - {S}_{{control}_{h}}} \\ {Fz}_{user} > {S}_{high} ‖ {Fz}_{user} < {S}_{min} : {S}_{user} = 0\end{matrix} ]$$

For this balance, the force measurement in the z direction is used to determine the user's stability, in which the values beyond the low and high thresholds specify that the user is not being properly supported by the walker. The values for S_{control}l, S_{control}h, Sₘᵢₙ, and Sₘₐₓ are determined experimentally for users based on their comfort and preference. The final desired velocity V_{ifinal} in Cartesian space is obtained using the below equation:

$${V}_{{i}_{final}} = {V}_{i} \cdot {S}_{user}$$

### Hardware Architecture

FIG. 10 illustrates the fully integrated walker with mechatronic components. The microcontroller chosen for this system is ESP32 due to its ability to have multiple serial communications. The main PC and ESP32 communicate to the AK10-9 motors (from T-motors) by sending motor commands and receiving encoder feedback via the SN65HVD230 CAN board (from AITRIP). Another ESP32 board communicates via serial to receive information from the RP LiDAR 1A scanner (from SLAMTEC). The RP LiDAR has a rotation frequency of 10 Hz with a total of 1450 sample points per rotation. A 48V battery powers the whole mechatronic system with a power converter to step down the voltage for the PC and DAQ device (from National Instruments).

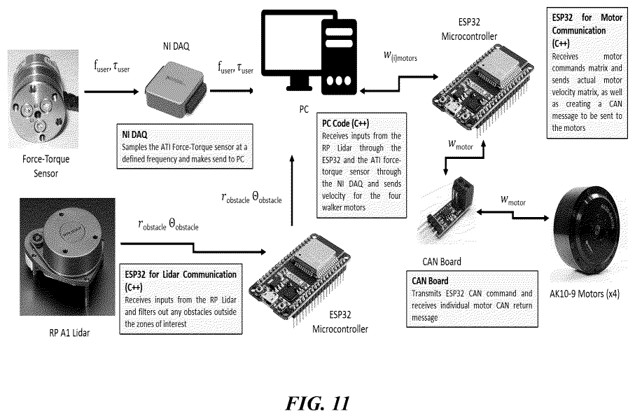

FIG. 11 represents the components and configuration schematic of the walker's mechatronic system used to receive sensory information and control the motors. FIG. 11 is configuration of the mechatronic system for the smart walker with individual component outputs for the ATI force torque sensor, RP LiDAR, PC, ESP32 microcontrollers, and CAN board.

The software programming is conducted in C++ on the PC using Visual Studio Code due to its integration with PlatformIO IDE for the microcontrollers. The CAN messages may be required to control the AK10-9 motors and instructions for serial communication with the RP LiDAR sensor.

### Software Architecture

The main C++ code is split into 3 threads to allow for processes of acquiring and sending data for the potential field generation and admittance controller calculations. The “Main Thread” has a computation time of 50 ms due to the multiple matrix calculations needed to update the potential field matrix. The communication threads with other sensors and motors both have a sampling rate of 8 ms. The RP LiDAR delivers the angle and distance of all surfaces scanned by the rotating laser. This information is then transformed into the x-y coordinate system considering the walker's front center as the origin.

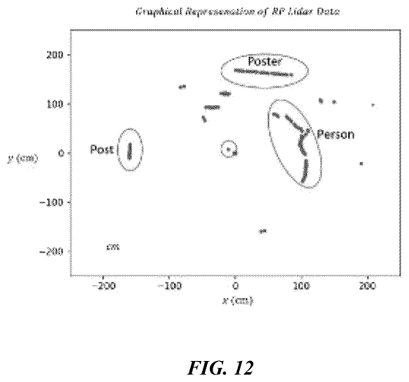

FIG. 12 illustrates an experimental result of the processed data from the RP LiDAR showing scanned obstacles around the sensor. Thereby, representing graphical representation of data received from the RP LiDAR sensor and processed to create a plot of obstacles around the sensor.

The code uploaded on the ESP32 pre-filters the LiDAR data to ensure that only information that is required, which is the obstacle location within the RZs zone, is transmitted to the main PC code to reduce the data for serial communications. The potential field generation portion of this code resets the obstacle distance matrix to accept updated values at the frequency of the main thread. The potential field generation is performed to update the potential field matrix. Once the potential field matrix has been generated, the “Potential Field Force Variable” mentioned in FIG. 13 is uploaded.

FIG. 13 represents architecture of the developed mechatronic systems, the executed tasks, and signal transmission between different component (including a PC, microcontrollers, DC motors, force sensor, and LiDAR scanner).

The “Velocity Output Algorithm” takes in the desired speed input from the user based on the admittance control and the “Potential Field Force Variable”. The velocity variable is updated every time new values are generated by the potential field.

### Experimental Evaluations

All components of the smart walker were integrated into a test bench first to conduct troubleshooting. The main experiments were conducted to evaluate the walker's performance in tracking desired velocities.

Admittance Control Experiments: In these experiments, a user attempted to move the walker in the desired direction as well as simulated instability. The following values were determined through various experiments based on a static walker; S_{control}l=−75 N, S_{control}h=−95 N, Sₘᵢₙ=−60 N, Sₘₐₓ=−110 N, T_{control}=10 Nm and Tₘₐₓ=30 Nm.

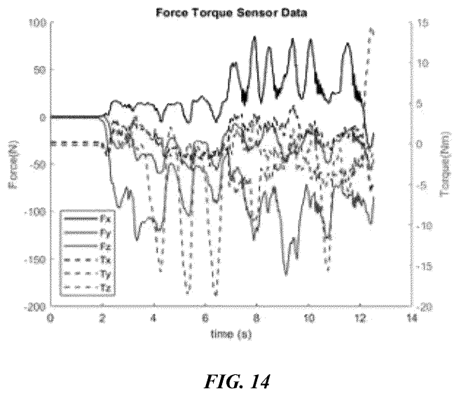

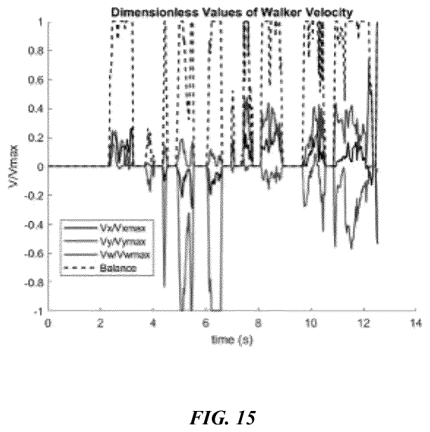

FIG. 14 illustrates the force and torque values measured by the ATI sensor under the handlebar. FIG. 15 represents the obtained values for the desired velocity were processed and obtained. The user attempted to apply the torques Tₓ and T_{y} and the force F_{z} to move the walker. This is due to the geometry of the walker's handle and the user stability requirements resulting in lying down on both arms and guiding the walker by torques.

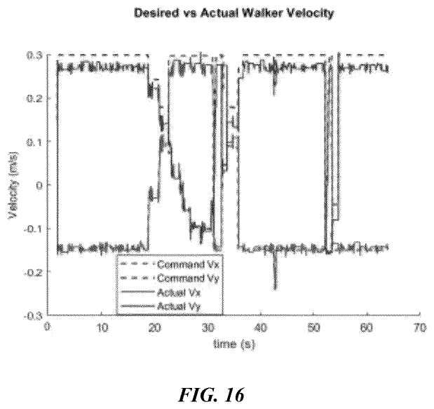

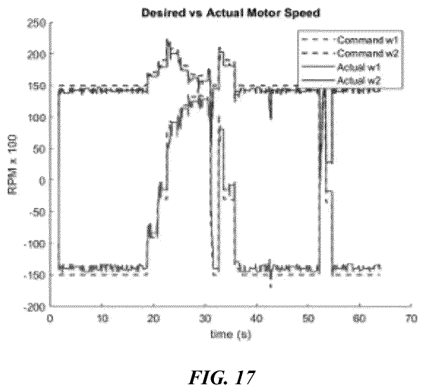

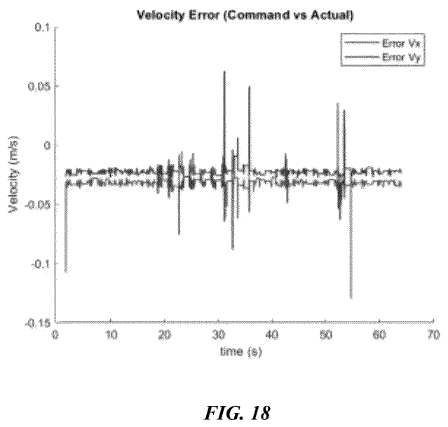

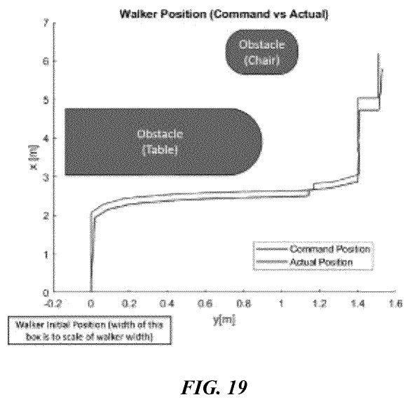

Obstacle Avoidance Experiments: With a constant desired velocity as the output of the admittance model, various tests were conducted in order to assess the performance of the potential field generation and the motor speed control. FIGS. 16, 17, 18 and 19 demonstrate the results of speed variation in Cartesian and wheels spaces, starting with an input velocity of 300 mm/s in the y direction. The speed controller's gain of the DC motor is adjusted on K_{d}=3 and the sampling time of 10 ms is employed in these experiments. As shown in FIG. 18, the average errors between the desired and actual velocities are 20.2 mm/s in the y direction and 2.9 mm/s in the x direction. FIG. 19 shows a graphical representation of the walker in Cartesian space and the performance of obstacle avoidance by online modification of the walker's speed as expected.

An admittance control method and an obstacle avoidance strategy were developed and tested for a motorized assistive walker. For this purpose, the mechatronics hardware and software were developed to generate and execute a continuous real-time motion trajectory for the smart walker, responsive to both user interaction force and the spatial distribution of scanned obstacles. By integrating components such as mecanum wheels, DC motors, microcontrollers, a rotating LiDAR scanner, and a mini-PC powered by an onboard battery, the obstacle avoidance algorithm and admittance control strategy were implemented. Through experimental evaluations, the autonomous walker's capabilities are demonstrated in generating the desired velocity in response to the user's physical interaction, navigating surrounding obstacles, and real-time adjustment of the motion trajectory. In future work, online mapping of the environment using a depth camera and image processing can be integrated into the control system. Moreover, user studies will be conducted to assess the effectiveness of this smart walker in assisting people with disabilities and navigating different environments.

### Design and Development of the Walker

Firstly, a spreadsheet was created to comprehend the physics underlying walker stability and determine the walker's dimensions by computing the walker tipping index and maintaining its value below 0.5. Furthermore, the pipe's bending stiffness and strength were calculated to finalize its dimensions. After extensive research to understand the physics behind a walker's stability, the “Walker Tipping Index” (WTI) concept was introduced and implemented to provide structural stability in future conceptual designs. To finalize the walker's overall dimensions, we need to calculate the ratio of the applied moment to the resistive moment due to the user's applied force on the handlebar.

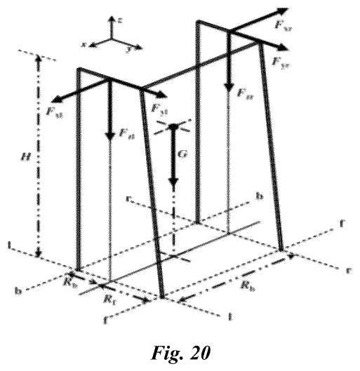

FIG. 20 represents the calculated tipping forces in reference to a Free Body Diagram. For forward and backward tipping calculation the following formula is used:

$$WTI = \frac{( {F}_{yl} + {F}_{yr} ) \times H}{( {F}_{zi} + {F}_{zr} + G ) \times {R}_{zi}}$$

Wherein, if the summation of left (F_{yl}) and right (F_{yr}) forces in the y direction (F_{yl}+F_{yr}) is greater than 0, the walker will tend to tip forward. For forward tipping R_{zi} is equal to R_{f}. And if the summation of left and right forces in the y direction (F_{yl}+F_{yr}) is less than 0 the walker will tend to tip backward. For backward tipping R_{zi} is equal to R_{b}.

For left and right tipping calculations we use the following formula:

$$WTI = \frac{( {F}_{xl} + {F}_{xr} ) \times H}{( {F}_{zi} + \frac{G}{2} ) \times {R}_{b}}$$

Wherein, if the difference of left (Fₓₗ) and right (Fₓᵣ) in the x direction (Fₓₗ-Fₓᵣ) is greater than 0, the walker will tend to tip leftward. For leftward tipping F_{zi} is equal to F_{zr}. And if the difference of left and right forces in the x direction (Fₓₗ-Fₓᵣ) is less than 0, the walker will tend to tip rightward. For rightward tipping F_{zi} is equal to Fₓₗ

In order to design a stable walker structure the walker tipping index must be below 0.5 which indicates that the applied moment on the walker is lower than the resisting moment of the walker structure which make the walker stable. “WTI” greater than 0.5 will have a tendency to tip in the applied force direction. From research regarding applied force by an average user on the right and left handle, it was found that 20% of the total weight of the user is applied on each handle at maximum loading or falling conditions. Using this, Table 1 was created to distribute the applied maximum force by the user in x, y, and z directions, with F_{z} (force in the z axis) taking up 60% of the total applied force by that arm.

TABLE 1
Distribution of forces in x, y, and z directions of
respective left and right arms for a 100 kg person.

| | Total | | | |
|---|---|---|---|---|
| Limb | Force | F_{y} | Fₓ | F_{z} |
| Right arm | 200N | 40N | 40N | 120N |
| Left arm | 200N | 40N | 40N | 120N |

The primitive dimensions are input for Table 2 which can be changed iteratively for better tipping ratios for maximum structural stability. Table 3 calculates the forward-backward tipping index and right-left tipping index using the equations (2) and (3) respectively.

TABLE 2
Dimensions of the walker's components:

| No. | Segment | Dimension |
|---|---|---|
| 1 | Height | 41 in |
| 2 | Front (R_{f}) | 16 in |
| 3 | Back (R_{b}) | 16 in |
| 4 | Track width (R_{b}) | 24 in |

TABLE 3
WTI values for different tipping directions

| No. | Tipping Direction | Value |
|---|---|---|
| 1 | Forward | 0.466 |
| 2 | Leftward | −0.078 |
| 3 | Backward | 0.466 |
| 4 | Rightward | −0.078 |

The main objective of the Walker tipping Index calculation is to optimize length, width, and height of the walker while ensuring that the “Walker Tipping Index” (WTI) remains below 0.5 and minimizing it while meeting the maximum dimensional limit for user's appropriate gait movement in the walker and accessibility of the walker in different locations. The WTI serves as a measure of the ratio between the applied moment and the resistive moment needed to move the walker. To prevent tipping, the generated directional moment must be always less than half of the applied moment. Using the dimensions derived from the WTI spreadsheet calculator and benchmarking the available rollators in the market, the initial Computer-Aided Design (CAD) was made using the SOLIDWORKS weldments feature.

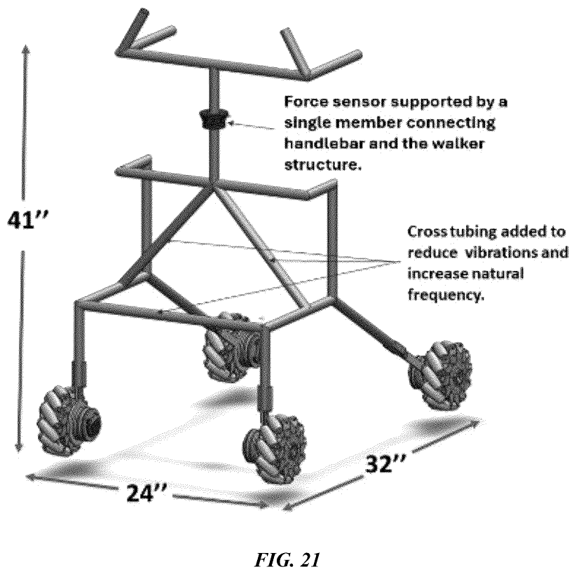

FIG. 21 shows the dimensions taken from the center of the walker as calculated by the spreadsheet calculator. FIG. 21 shows the Final walker design with force sensor only supported by user's applied force via the handlebar, with added cross pipes for a stiffer structure that has a high natural frequency.

The handlebars are directly linked to the force sensor, and this would yield the best results as required for the controller. Once the walker design was finalized Modal analysis was also performed.

In order to mitigate vibration's impact from the motor and gait movement of the user it is critical to determine the natural frequency of the walker, which can then be compared to the natural frequency of the motor to avoid resonance effects. The previous design's natural frequency was found to be relatively low after performing a Modal analysis on it. The FEA section contains information on the specifics. New members were added to increase the stiffness and, as a result, the natural frequency of the structure was increased because it was realized that the current design would vibrate due to minute bumps and path irregularities.

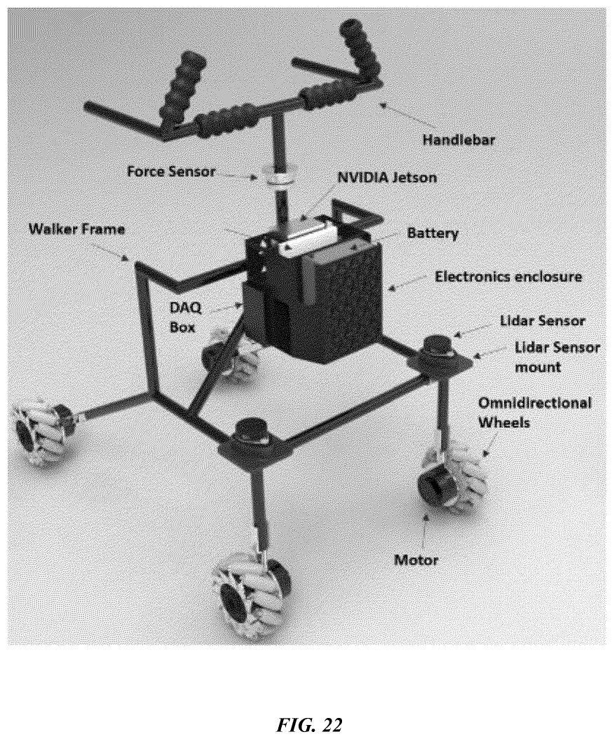

In FIG. 22, one can see the new members (pipes) connected from the center of the top bracing pipes to the middle of the side planar pipes and a cross member connecting the two pipes in the front. To develop a robust motor mount assembly involved de-signing a mount with a Factor of Safety exceeding 3 against all loading forces and motor torque. Utilizing 6061-T6 Aluminum with a 6 mm thickness, the mount was precision-manufactured via CNC. Complementing the assembly, 3 mm thick steel mounting clips were designed and produced. Integration employed 2 M5 flat head screws with corresponding nuts.

FIG. 22 displays the finalized assembly of the robotic walker, encompassing motors, omnidirectional wheels, sensors, controller box, force sensor, and the walker frame. FIG. 22 is a rendered CAD model of the walker with omnidirectional wheels, hub motors, electronics enclosure containing battery and electronics, LiDAR sensors and their mounts, along with handlebars for holding the walker from multiple locations. This design prioritizes lightweight construction while ensuring ample structural and postural stability, preventing tipping under normal loading conditions.

### Finite Element Analysis of the Structure

Modal analysis was conducted to assess and compare the stiffness of both the previous and final designs in terms of vibration reduction. Additionally, the natural frequency of the design was compared with the natural frequency of the motor, which was determined to be 840 Hz based on the provided motor data. This comparison was crucial to avoid resonance effects. To find out the natural frequency and to understand the behavior of the structure under vibration, Modal analysis on SOLIDWORKS was performed. Since the motor and the wheel assembly would be mounted on the bottom part of the walker, it was important to analyze the modes that emphasized the bottom members (pipes). It was found Mode 4 and Mode 5 were optimum for understanding the vibration behavior of the bottom members.

The natural frequency of the previous design in mode 4 came out to be 10.271 Hz and the natural frequency in mode 5 is 11.819 Hz which is still low for a structure that will have weight and motors attached to its bottom. Hence adding cross-members was crucial, and new pipes were added as mentioned in the design section. Frequency analysis was again performed to verify the increase in Natural Frequency. The natural frequency in the case of mode 4 came out to be 38.839 Hz, which is drastically more than the natural frequency in the previous design. Similarly, the natural frequency in mode 5 is 41.437 Hz, which is acceptable for a structure that will have weight and motors attached to its bottom. Hence adding cross members worked out for increasing the stiffness.

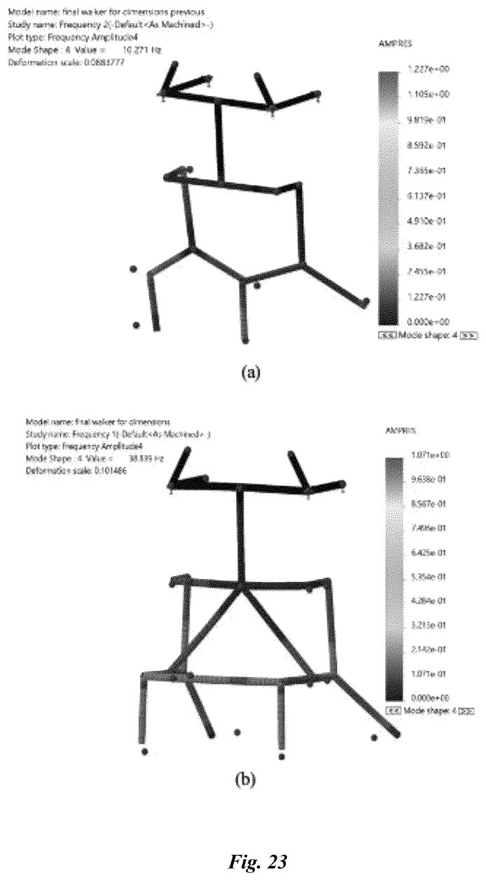

A comparison of mode 4 frequency between the previous and new design can be seen in FIGS. 23(a)-(b), where it is clear that the natural frequency increases 4 times after the addition of cross members to increase the structural rigidity of the walker. FIGS. 23(a)-(b) shows comparison of natural frequency between old and new design with added pipes for structural rigidity where, (a) Natural frequency of the old walker structure is 10.271 Hz, (b) Natural frequency of the new walker structure is 38.839 Hz.

### Symmetric and Asymmetric Loading Analysis

In a rigorous examination of the walker under a worst-case scenario, a load equivalent to 1.5 times the weight of a human (100 kg) was systematically applied downward onto the handle-bar to evaluate its structural integrity when a person applies all of their weight onto the handlebar, simulating falling forward and stabilizing manually. This investigation aimed to determine the strength of the structure and establish a Factor of Safety, an essential metric for ensuring design reliability. The boundary conditions entailed fixing all four bottom motor mounting points, creating a stable foundation. A uniform load of 1500 N was ap-plied to each part of the handlebar in the downward direction, mimicking extreme real-world conditions. The finite element analysis revealed a Von Mises Stress of 176.2 MPa on the structure, well below the yield strength of the AISI 4130 material (460 MPa). This result yielded a Factor of Safety of 2.6, an acceptable margin of safety in the worst-case scenario.

In conducting a comprehensive analysis of the walker under a worst-case forward tipping scenario, a load equivalent to the weight of a human (100 kg) was applied to the handlebar. This evaluation aimed to assess the structural strength and establish the factor of safety, crucial parameters for ensuring the robust-ness of the design. The boundary conditions involved fixing all four bottom motor mounting points, while a load of 1000 N was applied on right side of the handlebar in downward direction. The finite element analysis revealed a Von Mises Stress of 195.2 MPa on the structure, demonstrating a stress magnitude below the yield strength of the AISI 4130 material, which stands at 460 MPa. This outcome yields a Factor of Safety of 2.4, affirming an acceptable margin of safety in the worst-case scenario where an individual applies a load three times their body weight in the forward direction. The calculated Factor of Safety underscores the structural soundness of the walker, providing confidence in its ability to withstand substantial loads while maintaining integrity.

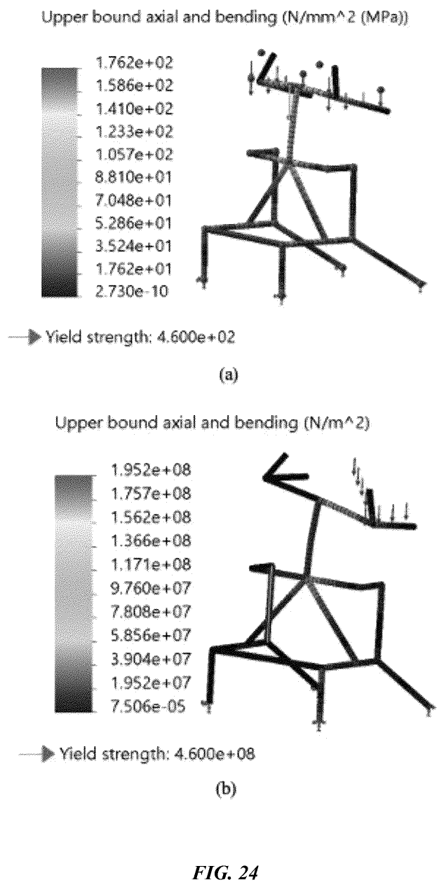

FIGS. 24(a)-(b) displays the resultant contours for worst-case scenario Analysis. FIGS. 24(a)-(b) shows a comprehensive structural analysis where (a) 1500 N force acting downwards on the handlebar, Von Mises stress is 176.2 Mpa (FoS is 2.6), (b) 1000 N force acting forwards on the handlebar, Von Mises stress is 195.2 Mpa (FoS is 2.4).

### Buckling and Torsional Analysis

In the pursuit of structural stability, a buckling analysis was systematically conducted to ascertain the load factor—an essential metric representing the ratio of the critical load for buckling to the applied load on the structure. This analysis was meticulously tailored to address a worst-case scenario, wherein a load equivalent to three times the weight of the individual was applied to the walker. The boundary conditions were rigorously defined with all four bottom motor mounting points fixed, and a down-ward load of 1000 N was uniformly applied to each part of the handlebar. The computed load factor is 5.94, which signifies that the structure possesses the capability to withstand a load nearly six times greater than the applied worst-case scenario load with-out succumbing to buckling. This result underscores the inherent rigidity of the structure against buckling in the given use case scenario, substantiating its resilience and structural integrity under the specified loading conditions.

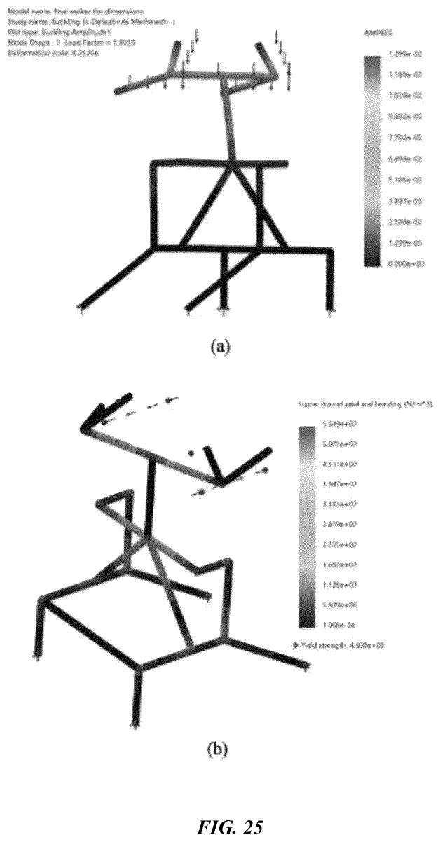

In a focused exploration of the torsional rigidity of the central member within the walker structure, a force of 200 N was applied as a coupling moment on the handle, simulating a scenario where a person exerts a 20 kg load on both sides of the handlebar in opposing directions, thereby imparting torque on the walker. The boundary conditions were rigorously specified, with all four bottom motor mounting points fixed, and the coupling force of 200 N was strategically applied on the edge of the walker. The finite element analysis disclosed a Von Mises Stress of 56.39 MPa on the structure, a stress magnitude comfortably below the yield strength of the AISI 4130 material, set at 460 MPa. This outcome yields an exceptionally high Factor of Safety of 8.2, indicating a substantial margin of safety. The calculated Factor of Safety underscores the remark-able structural rigidity of the central member in the face of ap-plied torsional forces. Refer to FIG. 25 to see additional FEA results done to determine the Torsional rigidity and Buckling Load factor.

FIGS. 25(a)-(b) represents additional analysis to assess the rigidity of the structure where (a) Buckling Analysis performed on handlebar when it bears triple the person's weight (Load Factor=5.94), (b) Torsional force couple of 200 N on handlebars, Von Mises stress is 56.39 Mpa (FoS is 8.2).

### Fabrication and Prototyping of the Walker

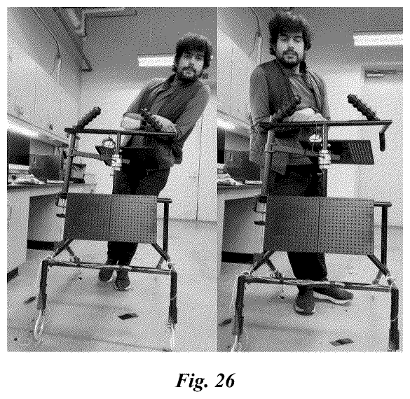

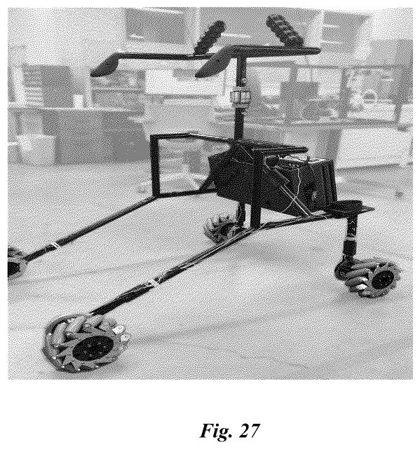

Concurrently with the design phase, extensive market re-search informed the selection of components to be integrated into the walker frame for assembly completion. A thorough ex-amination of available force sensors in the ATI company's cat-alog led to the choice of the Mini 58 sensor based on specific force requirements. Similar research methodologies were employed in the selection of sensors and motors. The initial stage involved cutting AISI 4130 pipes to desired lengths for various walker frame members. Subsequently, planar sub-assemblies were welded together in a jigged 3D configuration using TIG welding. Following the completion of planar surfaces, cross members were welded to form the 3D framework of the walker. Upon determining the optimal length of motor mounts, mounting clips were welded onto the pipes, resulting in the assembly's readiness for motor installation. Simultaneously, the handlebar, including 3D-printed grips in the final prototype, was manufactured. All manufactured and welded components were assembled to assess the assembly's fit and understand tolerance effects. A conclusive stability test, incorporating all mounting components, was successfully conducted through a test run. The walker proved highly stable and did not tip forward-backward or leftward-rightward even after loading the entire body weight on each side, which can be seen in FIG. 26 and this verified the WTI considerations taken during the design phase. FIG. 27 shows the final assembly and manufactured walker with all the components.

FIG. 26 represents Postural stability test on the walker structure to validate the WTI calculations and structural strength. FIG. 27 is the Assembled walker prototype with all the mechanical and electrical components.

The development of an intelligent robotic walker was meticulously carried out through a systematic process, focusing on critical aspects such as stability, structural strength, and user comfort. The initial phase involved precise calculations of fundamental dimensions using a Walker Tipping Index (WTI) spreadsheet calculator, optimizing dimensions to maintain a WTI below 0.5 for stability in all directions. The robustness of the force sensor, vital for user interaction, was ensured by considering a maximum load of 100 kg. After evaluating various force sensors, the “mini-58” sensor from “ATI” was selected for its optimal performance characteristics.

In the walker's structural design, rigorous weight optimization and stability were considered through symmetric-asymmetric loading, buckling, and modal analyses. The factor of safety (FoS) was maintained above 2 according to different analyses, ensuring the walker's structural integrity in critical loading conditions. Advanced techniques were employed in manufacturing, including Tungsten Inert Gas (TIG) welding for precise and sturdy construction of structural components. Electronic en]closures and LiDAR mounts were 3D printed with carbon fiber-infused PETG for lightweight durability, while motor mounts were CNC machined for optimal performance. A rigorous physical stability test validated the Walker Tipping Index (WTI) calculations, confirming postural stability and structural strength. As part of the future work, redesign of the walker structure will be done in order to reduce the weight and increase the structural rigidity using composite materials like carbon fiber. To improve the gait performance and mapping of the environment motion planning will be enabled in the controls of the walker to enhance object detection and fall prevention capabilities.

The instant description is provided as an enabling teaching of the disclosure in its best, currently known aspect. Those skilled in the relevant art will recognize that many changes can be made to the aspects described, while still obtaining the functional results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the instant description is provided as illustrative of the principles of the present disclosure and not in limitation thereof.

What is claimed is:

1. A walker comprising:
one or more LiDAR sensors configured to perform range finding and obstacle detection; and
an autonomous walker controller configured to:
generate a motion trajectory for electric motors based on a user's surroundings, body posture, and intended movement;
automatically guide the walker around obstacles with the motion trajectory, even if in conflict with the intended movement;
guide the walker into a more supportive position with the motion trajectory if the user is at imminent risk of a fall;
stop all movement if a use scenario becomes unsafe;
identify a fall by estimating a relative position of a user's center of mass; and
generate a profile of a user's habitual movements and preferences to personalize the generated motion trajectory.

2. The walker of claim 1, further comprising a rigid frame having a left side and a right side.

3. The walker of claim 2, further comprising a caster wheel attached to a front portion of each of the left and right sides of the rigid frame.

4. The walker of claim 2, further comprising a motorized wheel assembly attached to a rear portion of each of the left and right sides of the rigid frame, each motorized wheel assembly comprising an electric motor with a driven output coupled to a wheel.

5. The walker of claim 4, further comprising sensors configured to detect rotational position and speed of the electric motors.

6. The walker of claim 2, further comprising an adjustable armrest with an integrated grip and a plurality of force sensors attached to each of the left and right sides of the rigid frame.

7. The walker of claim 1, further comprising brake levers physically coupled to brakes on wheels and sensors configured to detect an amount of brake actuation.

8. The walker of claim 1, further comprising one or more cameras oriented toward the user to monitor user posture, gait, and balance.

9. The walker of claim 1, further comprising one or more cameras oriented toward an environment surrounding the walker to monitor type and position of obstacles.

10. The walker of claim 1, further comprising user interfaces for control comprising one or more of: screens to display control and user interfaces, physical buttons, microphones, speakers, and LED lights.

11. The walker of claim 1, further comprising a seat permanently fixed between left and right sides of a rigid frame.

12. The walker of claim 1, further comprising a stowable seat permanently fixed between left and right sides of a rigid frame.

13. The walker of claim 1, further comprising stowable foot rests configured to support weight of the user while standing on the walker.

14. The walker of claim 12, further comprising a stowable foot rests of claim 13.

15. The walker of claim 1, wherein the autonomous walker controller is further configured to monitor sensor data to map surroundings of the walker and track body posture of the user.
